(19) United States
(12) Patent Application Publication
(10) Pub. No.: US 2024/0382708 A1
(43) Pub. Date: Nov. 21, 2024

(54) Removable Tracheostomy Cannula Monitor

(71) Applicants: Gil Zoizner-Agar, Tel Aviv-Yafo (IL); Itzhak Pomerantz, Kefar Sava (IL); Michael Nagler, Tel-Aviv (IL); Roy Zahor Chehanover, Tel-Aviv (IL)

(72) Inventors: Gil Zoizner-Agar, Tel Aviv-Yafo (IL); Itzhak Pomerantz, Kefar Sava (IL); Michael Nagler, Tel-Aviv (IL); Roy Zahor Chehanover, Tel-Aviv (IL)

(21) Appl. No.: 18/661,815

(22) Filed: May 13, 2024

Related U.S. Application Data
(60) Provisional application No. 63/466,358, filed on May 15, 2023.

(51) Int. Cl. A61M 16/04

(52) U.S. Cl. CPC A61M 16/0465; A61M 2205/0294; A61M 2205/18; A61M 2205/3553

(57) ABSTRACT

A removable tracheostomy cannula monitor for monitoring, displaying, and alerting on medical situation of a patient using a cannula. The cannula monitor connects to a cannula. In some embodiments, the cannula monitor also connects to an additional medical device, such as an external ventilation unit. Also in some embodiments, the cannula monitor sends data to a smart phone and/or the patient's electronic medical/health records for processing, display, and communication with a care giver.

### BACKGROUND

Various means are known in the art for providing an alert during an emergency for a patient using a cannula after a tracheostomy. These means are needed as a patient using a cannula has a high risk of an adverse medical event, specifically a respiratory accident. As patients in such condition often have impaired vocalization control, their ability to call for help is limited.

Among others, the following patents describe solutions to such problem: U.S. Pat. No. 5,367,292 to Szoke et al., U.S. Pat. Pub. No. 2011/0144514 to Booker, Int'l Pat. App'n Pub. WO 00/48510 to Weil et al., and U.S. Pat. Pub. No. 2015/0209532. Prior art solutions such as these are “permanent” and intervene with the design of the cannula. Being permanent, they are cumbersome to medical staff when a cannula needs maintenance and limit the selection of possible cannulas for the patient. Other limitations of prior art solutions are: the requirement that the patient be mechanically ventilated and/or have a cuff between the outer rim of an endotracheal tube or tracheostomy cannula and the trachea; the lack of monitoring other vital signs that are indicative of the patient's condition, and thus are more prone to false alarms or cannot provide sufficient information regarding the effect on the patient's overall condition; and no allowance of some prior art solutions for providing information by the patient or caregiver, which can give a remote physician important anamnestic details.

It would be very desirable to have a removable universal means for monitoring the vital signs of a patient using a cannula, which would fit any standard cannula and enable a medical practitioner to easily and safely remove these means from the cannula to have free and normal access for cannula maintenance and for treating the patient. However, the present inventors know of no such means.

The following terminology is used in the present disclosure:

Cannula (sometimes referred to as “tracheostomy cannula”)—a curved tube inserted into a tracheostomy stoma (hole made in the neck and windpipe (Trachea)). Tracheostomy tubes vary in features and intended use.

Cannula collar (ribbon or tie)—a strap around the neck of a patient, which secures the tracheostomy cannula.

Cannula front unit—a removeable device that can be mechanically and reversibly mounted on the front end of a tracheostomy cannula.

Cannula back unit—a device mounted on the back of a cannula collar or distributed along the cannula ribbon, connected to the cannula front unit and providing battery power, communication circuitry and sensors in touch with the patent's skin.

Removable—can be reversibly attached and detached by a simple mechanical operation such as plugging, threading/twisting, and locking a bayonet connector.

Vital signs—measurements of the body's most basic functions. The main vital signs routinely monitored by medical professionals and health care providers include body temperature, pulse rate, respiration rate, oxygen saturation, and blood pressure

### SUMMARY

The present invention may be embodied as a stand-alone device that contains its own energy source and communications module, contains sensors that measure parameters of the inhaled and exhaled air along with other vital signs measured from the breath and neck skin around the cannula, and delivers the measurements to a processor (included in the device in some embodiments) that analyses them in real time and generates an alert if they indicate a problem.

The device as a front unit that is shaped like a short tube that can be connected to the external opening of a cannula similarly to how other medical devices are designed to plug into the cannula. One example is an external ventilation device. The front opening of the tube-shaped device, in a preferred embodiment of the invention, repeats the shape of the cannula opening, so it can fit devices that are designed to be connected to a cannula. Compatibility with other cannula openings is possible as well with the other side still being compatible with mechanical ventilation systems, so the device can be joined between a standard cannula and external devices.

In a preferred embodiment, the device is self-contained, and it includes a power source, such as batteries, and communication circuitry.

In another preferred embodiment, the device contains sensors that sense the flow of air, while other sensors, batteries, and circuitry are packaged in a second unit, mounted along the cannula collar, and connected to the front unit by a cable.

The front unit can be easily removed from the cannula, thereby exposing it and enabling standard routine treatment, such as cleaning occlusions.

The device has means for sensing at least some of the capacity (volume of air flowing through the device in one breath), flow rate, temperature, humidity, CO₂ concentration, and audio content (spectrum of the audio signals created by the breathing action) of the air flow in both directions. The collar that typically secures the cannula around the neck of the patient can contain additional skin sensors, such as a PPG (photoplethysmography, i.e., blood oxygen saturation) sensor, and necessary hardware, such as batteries and communication circuitry.

### BRIEF DESCRIPTION OF THE DRAWINGS

The invention is described below in the appended claims, which are read in view of the accompanying description including the following drawings, wherein:

FIGS. 1A-1D show a cross-section of a standard cannula with and without a removable front-end cannula monitor;

FIGS. 2A-2E provide various illustrations an example sensor for measuring the flow rate of the air in a cannula;

FIG. 3 is a flowchart of an algorithm for creating an alert;

FIG. 4 illustrates a simplified diagram of a system using the removable front end of the cannula monitor and a standard smart phone; and

FIGS. 5A-5C shows another preferred embodiment measuring the air flow in a cannula monitoring device.

### DETAILED DESCRIPTION

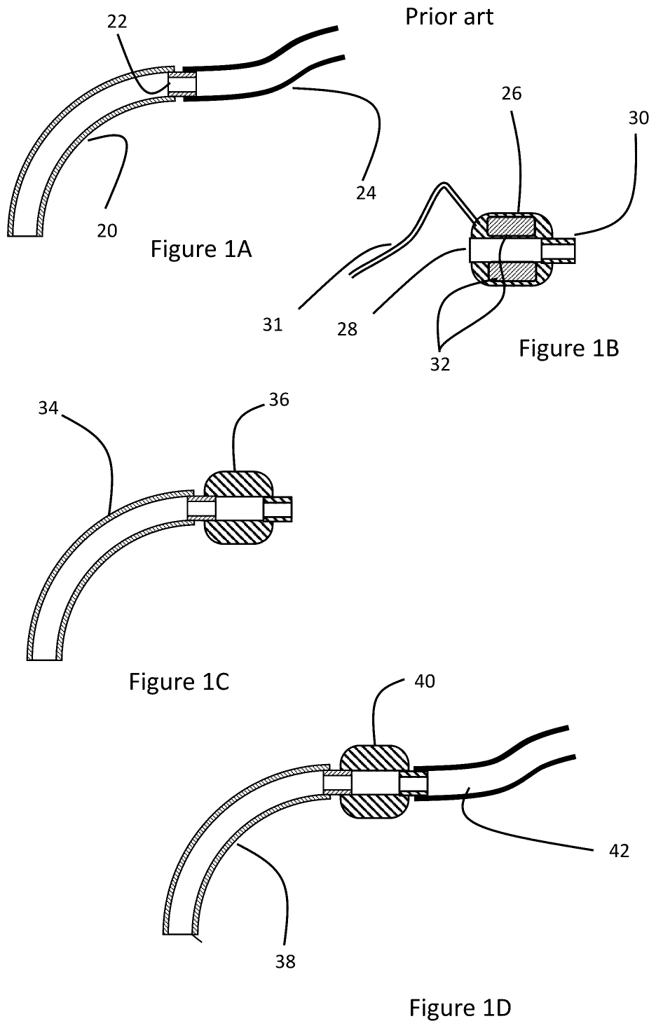

Attention is now called to FIGS. 1A-1D.

FIG. 1A shows a simplified ordinary prior art cannula 20, such as described in the John Hopkins Hospital internet page https://www.hopkinsmedicine.org/tracheostomy/about/types.html. The front opening 22 of the cannula is a tube with standard diameter and configured to fit attachable devices, such as external ventilation devices, one of them schematically represented at 24.

FIG. 1B shows a cross section of a simplified removable cannula monitor 26. It has an air inlet 28 that can be plugged to the standard cannula end and an air outlet 30 that can be connected to attachable devices such as external ventilation devices. At least one sensor 32 is installed inside cannula monitor 26 and around the air passage, measuring vital signs, such as, air flow direction, air flow speed, air temperature, air humidity, and concentration of CO₂.

The vital signs can be measured by sensors known in the art and available in the market. CO₂ sensors are described in https://www.infineon.com/cms/en/product/sensor/co2-sensors/?utm_source=GoogleCPC&utm_medium=GoogleAds&utm_campaign=as-css-en-gsg_cloudid_product_cloudid&gclid=CjOKCQiA0eOPBhCGARIsAFIwTs5-pmILBHC0zjnzWX3n_0f5XUZxRkDoSYOuHPQSCCMQvRxl_HxzEAwaAmLdEALw_wcB&gclsrc=aw.ds.

Humidity sensors are described in https://www.vaisala.com/en/measurement/humidity-dew-point-and-moisture?utm_medium=cpc&utm_source=google&utm_campaign=VIM-GLO-EN-HUM-Gen&gclid=Cj0KCQiA0eOPBhCGARIsAFIwTs56fcUW11EKJ8GhkSSeBLoXU KSNwLxQGkHtWUG2GSV_qPfEeHeSnwoaAhw9EALw_wcB.

Temperature sensors are described in https://www.directindustry.com/industrial-manufacturer/temperature-sensor-medical-applications-199357.html.

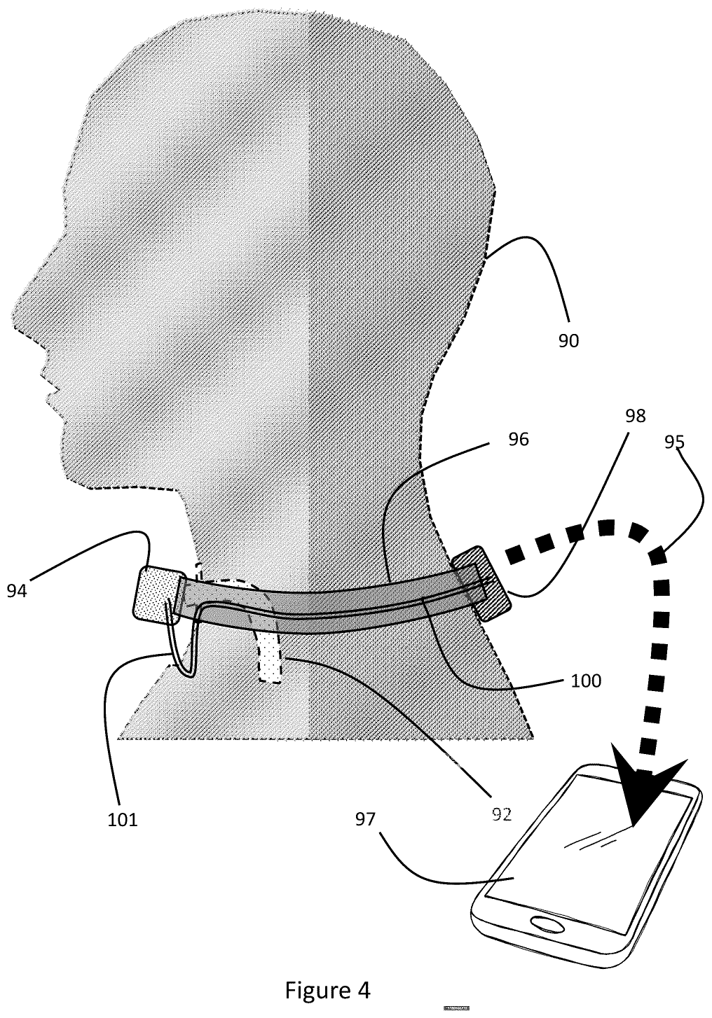

The cannula monitor can be wireless, having internal batteries and radio communication with a processor, such as that of a mobile phone. Alternatively, it can be wired 31 to a support unit wearable by the patient, such as that shown in FIG. 4 below. The support unit can hold the batteries, communication circuitry, and possibly a processor, thereby minimizing the size and weight of the cannula monitor.

FIG. 1C shows the cannula monitor 36 mounted on the cannula 34.

FIG. 1D shows a device 42, such as an external ventilation unit, connected to a cannula monitor 40, which is connected to a cannula 38.

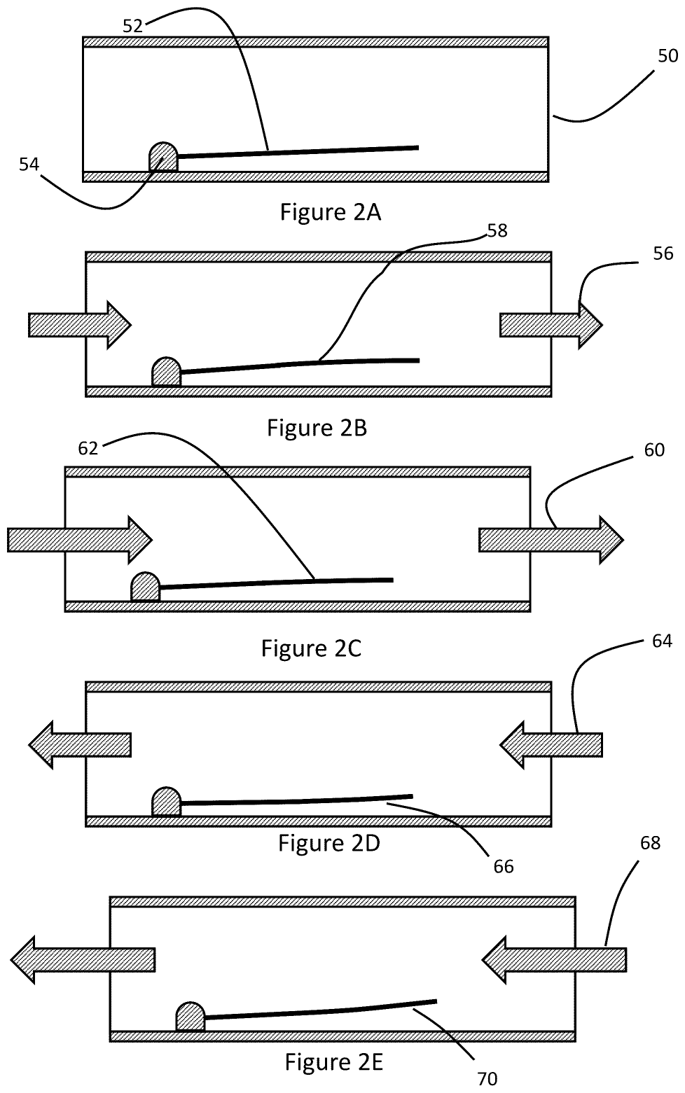

Attention is now drawn to FIG. 2A-2E showing a preferred embodiment of an air flow sensor in a cannula monitor. In FIG. 2A, a thin leaf 52 cut from piezoelectric transducer material, such as MCFT-27T-4.2AL-127, Multicomp Pro-PIEZO ELEMENT (https://il.farnell.com/multicomp-pro/mcft-27t-4-2al-127/piezo-element/dp/1801061?gclid=Cj0KCQiAi9mPBhCJARIsAHchl1y2zU5RB6aEPR oCnc01zaRsbZJMbhQqq5Fc9yTTIxy5n_ajxo9p3AUaAupHEALw_wcB&mckv=s_dc|pcrid|454051002933|plid∥kword∥match∥slid∥product|1801061|pgrid|10 5432025625|ptaid|pla-363010435644|&CMP=KNC-GIL-GEN-SHOPPING&gross_price=true), is held by a support 54 in the air passage of the cannula monitor 50. When there is no flow of air in the passage, the piezo electric transducer does not generate any voltage.

FIG. 2B shows a slow flow of air 56 from left to right, representing inhaling by the patient. Due to the diagonal position of the piezoelectric leaf 58, the flow of air will bend it downwards and a voltage will be generated.

FIG. 2C shows a faster flow of air 60 from left to right. The piezoelectric leaf 62 will bend further down and/or bend faster, thereby generating a higher voltage of the same polarity.

FIG. 2D shows a slow flow of air 64 in the opposite direction, representing an exhaling action by the patient, due to the diagonal position of the piezoelectric leaf 66, it will bend upwards and will generate a voltage of opposite polarity.

FIG. 2E shows a faster flow of air 68, and the piezoelectric leaf 70 will bend upwards more and/or bend faster, thereby generating a higher voltage.

As demonstrated, the voltage generated by the piezoelectric leaf is indicative of the momentary direction and the speed of the air flow. When the air flow ceases and the leaf elastically returns to its default position, a voltage in the opposite direction is generated, and by integrating the voltage over time the system can closely estimate the maximum speed and the volume of air while the user inhales and exhales.

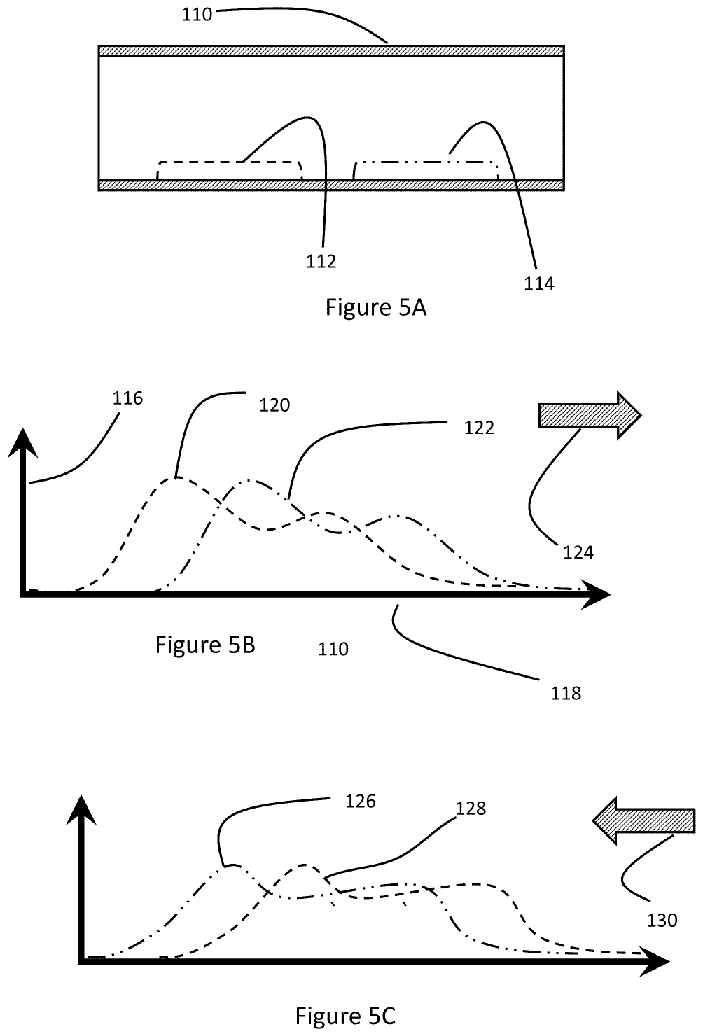

Attention is now drawn to FIGS. 5A-5C showing another preferred embodiment of measuring the air flow in a cannula monitoring device.

FIG. 5A shows air passage 110, which resembles that shown in FIG. 2A. Two piezoelectric leaflets 112 and 114 are mounted on the inner wall of the device one after the other along the air flow path. As in FIG. 2A, both piezoelectric leaflets 112 and 114 of FIG. 5A are secured at one of their edges while the other of the two edges are free to move up or down in accordance with the surrounding air flow. For clarity, though, they are represented as simple rectangles in the figure.

Due to Bernoulli's law, an upwards force is applied to the leaves when the air flows tangential to them, and the amount of the force will be indicative of the speed of the flow. The two leaves each have a voltage pattern, and those voltage patterns are correlated. The time difference of the voltage pattern waveforms is indicative of the flow speed.

FIG. 5B shows the voltage 118 of the signals over time 118 from both leaves. For clarity, the font of the line of the leaves in FIG. 5A is the same as the font of the chart in FIG. 5B. As can be seen in FIG. 5B, the trace 120 coming from leaf 112 is advanced in time compared to the trace 122 coming from leaf 114. A processor that cross-correlates the two signals, will determine that leaf 112 sensed the air flow before leaf 114 sensed it and will conclude that the air flow was left to right 124.

FIG. 5C also shows the voltage over time of both leaves. For clarity, the font of the line of the leaves in FIG. 5A is again the same as the font of the chart in FIG. 5C. As can be seen in FIG. 5C, the trace 128 coming from leaf 112 is lagging in time compared to the trace 126 coming from leaf 114. A processor that cross-correlates the two signals can determine that leaf 114 sensed the air flow before leaf 112 sensed it, and will conclude that the air flow was right to left 130.

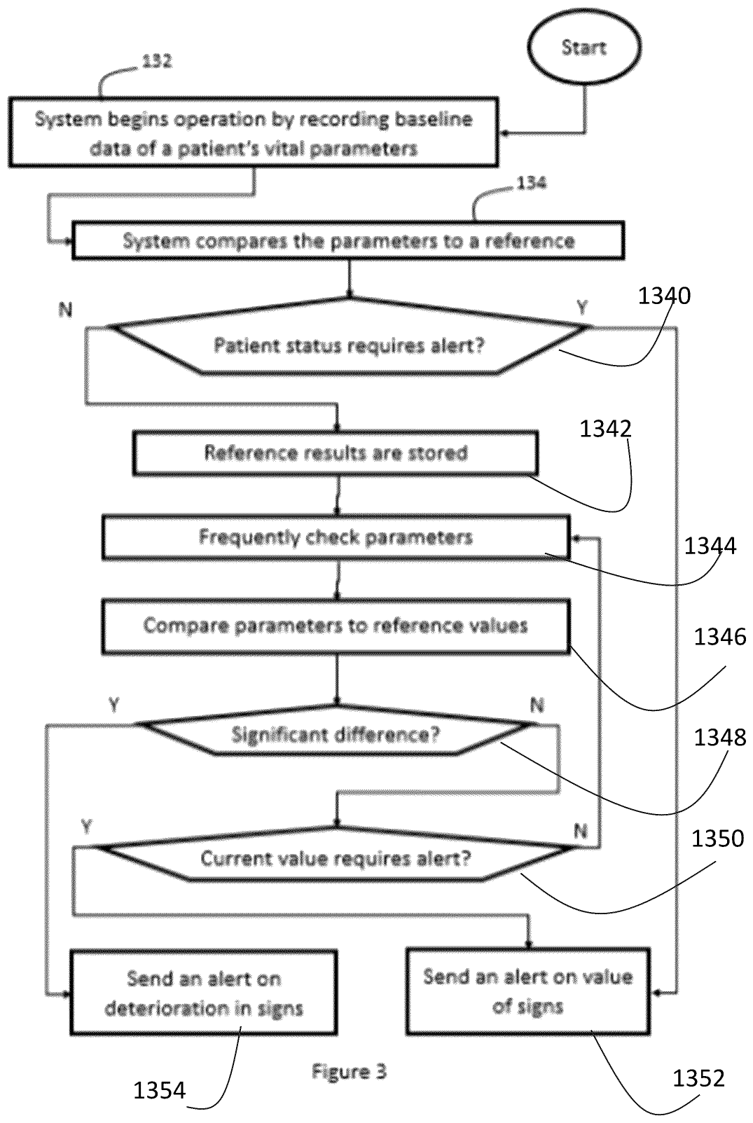

Attention is now drawn to FIG. 3, which provides a simplified flowchart of routine use of embodiments of the present invention. The system issues an alert to a care giver, in the hospital or at home, in any of two situations:
1. The value of some of the vital parameters exceed pre-determined thresholds; and
2. The value of some of the vital parameters suddenly deviates from the normal values of that patient.

Air flow properties can be measured by other means such as used in spirometers, as described in the Wikipedia article at https://en.wikipedia.org/wiki/Spirometer.

The flowchart in FIG. 3 shows the logic of the process in a preferred embodiment of the invention. As seen in the flowchart, alerts can be created on two situations:
1. The combination of measured parameters indicates an emergency that requires sending an alert to a care giver; and
2. Some of the measured parameters tend to significantly deviate from a reference measurement in which the patient appeared to be feeling well, and the deviation requires an alert to a care giver.

In a preferred embodiment of the invention, the patient can input his/her subjective self-assessment of his health by responding to a simple questionnaire, such as on his telephone, and a summary of his input is treated as one of the parameters for use in addition to the parameters that are measured by the sensors. The telephone's keyboard and display can be used for input/output with the patent, and the telephone's processor can run an application in the telephone's memory to execute the questionnaire.

In an embodiment, the system begins operation by recording baseline data of a patient's vital parameters 132.

Then, the system compares the data to a reference 134, resulting in two criteria for the system to issue an alert: meeting predetermined values of emergency, and detecting an increasing deviation from a given baseline.

Then, the system determines whether the deviation of the measured signals from the baseline signals meets pre-determined conditions for an “emergency state” 1340.

If the determination in step 1340 is affirmative, the system creates an alert message based on the deviated values of the signals, while providing the measured signals 1352

If the determination in step 1340 is negative, the system stores the measured signals 1342 and continues to measure the signals periodically 1344, compares the results to the base line data 1346, and if it detects an increasing deviation from the baseline signals 1348, the system sends an alert that the signals are increasingly deviating from the baseline, even if the system itself does not recognize the meaning and significance of this increasing deviation 1354. If the system does not detect an increasing deviation from the base line, it checks 1350 if the current values of the signals meet the predetermined emergency values, and if so, creates the emergency alert 1352.

Attention is now called to FIG. 4, showing a simplified layout of the system. A patient 90 using a cannula 92 wears a strap 96 to support the cannula and to keep it in place. A front part 94 of the cannula monitoring device is plugged into the front end of the cannula, and the front part 94 has sensors to monitor the inhaled and exhaled air. Measurement data are sent 95 to the patient's smart phone 97 for processing, display, and communication with a care giver.

A rear part 98 of the cannula monitoring device is mounted adjacent the neck of the patient and in contact with the patient's skin. The rear part 98 contains batteries and communication circuitry, as well as sensors that measure additional parameters from the skin, such as Galvanic Skin Response (GSR), pulse rate, and oxygen saturation.

The two parts 94 and 98 are connected by a multi-wire cable 100 that provides power to the front end and sends measurement signals to the rear end. The cable 100 has a slack portion 101 to allow removal of the front end from the cannula for servicing of the cannula and for treating the patient in a clinic. The system can detect the act of unplugging the monitor from the cannula and create a non-compliance alert to notify medical staff of unauthorized disconnections.

Having thus described exemplary embodiments of the invention, it will be apparent that ideas for various alterations, modifications, and improvements will readily occur to those skilled in the art. Alternations, modifications, and improvements of the disclosed invention, though not expressly described above, are nonetheless intended and implied to be within spirit and scope of the invention. Accordingly, the foregoing discussion is intended to be illustrative only; the invention is limited and defined only by the following claims and equivalents thereto.

1. A removable tracheostomy cannula monitor comprising:
an air inlet configured for attachment to a cannula;
an air outlet;
an air passage connecting the air inlet to the air outlet; and
at least one sensor inside the air passage.

2. The monitor of claim 1 further comprising:
a first unit; and
a second unit;
wherein the first unit is plugged into the cannula and the second unit is mounted on a cannula collar.

3. The monitor of claim 2, wherein the front unit has sensors configured to measure one or more of the following parameters: flow direction, flow speed, inhaled capacity, humidity temperature, and CO₂ concentration.

4. The monitor of claim 2, wherein the rear unit has sensors configured to measure one or more of the following parameters: galvanic skin response, pulse, and oxygen saturation.

5. The monitor of claim 2, wherein the rear unit is distributed along the cannula collar.

6. The monitor of claim 2 further comprising:
communication circuitry between the monitor and a mobile telephone.

7. The monitor of claim 6 further comprising:
a software application on the mobile telephone for at least one of storing, processing, displaying, and communicating information related to the parameters measured by the monitor.

8. The monitor of claim 6 further comprising:
an application stored in the mobile telephone to obtain a patient's subjective self-assessment of his health.

9. An emergency notification method for a patient wearing a tracheostomy cannula and a cannula monitor, the method comprising:
a. monitoring sensor data provided by the cannula monitor;
b. determining whether the sensor data exceed preset thresholds;
c. determining whether the sensor data differ more than a set amount from sensor data measured in a recent non-alerting situation; and
d. generating an emergency notification if the sensor data exceeds a preset threshold and/or the sensor data differ more than the set amount.
